(19) United States
(12) Patent Application Publication
(10) Pub. No.: US 2017/0073634 A1
(43) Pub. Date: Mar. 16, 2017

(54) METHOD OF INDUCING CALLUS, METHOD OF CULTURING CALLUS, METHOD OF INDUCING SOMATIC EMBRYO, METHOD OF REGENERATING PLANT, AND METHOD OF PROPAGATING PLANT

(71) Applicant: SUMITOMO RUBBER INDUSTRIES, LTD., Kobe-shi (JP)

(72) Inventors: Akari OKADA, Kobe-shi (JP); Sompong TECHATO, Hat Yai (TH)

(73) Assignee: SUMITOMO RUBBER INDUSTRIES, LTD., Kobe-shi (JP)

(21) Appl. No.: 15/242,907

(22) Filed: Aug. 22, 2016

(30) Foreign Application Priority Data
Sep. 14, 2015 (JP) 2015-180877

(51) Int. Cl. C12N 5/04; A01H 4/00

(52) U.S. Cl. CPC C12N 5/04; A01H 4/005; C12N 2500/10; C12N 2501/305; C12N 2500/76

(57) ABSTRACT

The invention provides: a callus induction method that efficiently induces callus from a tissue fragment from an isoprenoid-producing plant; a callus culture method that efficiently grows callus; a somatic embryo induction method that efficiently forms a somatic embryo; a plant regeneration method that can stably regenerate callus into plants; a plant propagation method that can stably propagate a plant without being affected by e.g. weather and seasons; and a method of inducing rooting in a mature embryo. The present invention relates, inter alia, to a method of plant regeneration that can stably regenerate callus into plants and a method of plant propagation that can stably propagate a plant.

### TECHNICAL FIELD

The present invention relates to a method of inducing callus, a method of culturing callus, a method of inducing a somatic embryo, a method of regenerating a plant, and a method of propagating a plant.

### BACKGROUND ART

At the present time, the natural rubber (a type of polyisoprenoid) used in industrial rubber products is obtained by the cultivation of rubber-producing plants, e.g. Para rubber tree (Hevea brasiliensis) of the family Euphorbiaceae or Indian rubber tree (Ficus elastica) among Moraceae plants; the laticifer cells in these plants biosynthesize natural rubber, and this natural rubber is collected from the plants by manual procedures.

Natural rubber for industrial applications is at present sourced almost entirely from Hevea brasiliensis. Moreover, it is used widely and in large amounts in a variety of applications as the main raw material for rubber products. However, Hevea brasiliensis is a plant that can be grown only in limited regions such as Southeast Asia and South America. Furthermore, Hevea brasiliensis requires about seven years from planting to becoming mature enough for rubber extraction, and the collection season is limited. The time period during which natural rubber can be collected from the mature tree is also limited to 20 to 30 years.

For the future, an increase in the demand for natural rubber, centering on developing countries, can be expected, while for the reasons indicated above it is difficult to greatly increase the production of natural rubber from Hevea brasiliensis. As a consequence, the exhaustion of natural rubber resources is a matter of concern, and there is a desire for improvements in the yield from Hevea brasiliensis.

With Hevea brasiliensis, seedling propagation is carried out by raising and growing seedlings provided by seeding to yield a rootstock and grafting buds obtained from clone plantlets to the rootstock. Since there are limitations on the buds obtained from clone plantlets, the mass propagation of improved clone plantlets is necessary in order to spread improved clones.

In addition, grafting, which is a conventional clonal propagation technique, can at the same time perpetuate the diseases present in the original tree and thus can result in the propagation of diseased seedlings. There is therefore a desire for a method that enables a stable plant propagation.

It is known that the productivity and quality can be altered through selection of the rootstock. Much of the rootstock in current use is seedling rootstock, and the genetic characteristics are not uniform and it is difficult to select excellent rootstock.

Further, since time is required to grow to a size that can be used as rootstock and since in seed propagation the genetic traits are more likely to be unstable, the use of general crossbreeding presents difficulties. Furthermore, the rootstock may exercise an effect on the graft and as a consequence the risk arises that a true clone plantlet will not be obtained.

In order to increase the amount of isoprenoid production in plants, methods are considered for plant improvement with the objective of, for example, improving the resistance to stress or increasing the amount of isoprenoid accumulated in the plants. Other possible methods for plant improvement are to use artificial crosses or mutations, but it is difficult to efficiently impart desired characteristics and the feasibility of these methods seems to be low. Due to this, it is considered that a cell engineering approach that involves the introduction of a target gene into plant cells to impart desired characteristics will be used for plant improvement.

When the cell engineering approach is used, it is then necessary to induce redifferentiation of the target gene-bearing plant cells into plants. In other words, plants must be regenerated from plant cells (for example, callus). While various investigations into tissue culture in plants have been carried out, there are almost no examples of investigations into methods of plant regeneration from callus from an isoprenoid-producing plant, and it has been difficult to stably regenerate callus into plants.

### SUMMARY OF INVENTION

### Technical Problem

The present invention solves the aforementioned problems, and objects of the present invention are to provide a callus induction method that efficiently induces callus from a tissue fragment from an isoprenoid-producing plant, a callus culture method that efficiently grows callus, a somatic embryo induction method that efficiently forms a somatic embryo, a plant regeneration method that can stably regenerate callus into plants, a plant propagation method that can stably propagate a plant without being affected by e.g. weather and seasons, and a method of inducing rooting in a mature embryo.

### Solution to Problem

As a result of intensive investigations, the present inventors discovered that a somatic embryo induced from callus can subsequently undergo stable germination and rooting and consequently a regenerated individual can be obtained. That is, it was discovered that, through the induction of a somatic embryo from callus, callus can be stably regenerated into plants and, further, the plant can be stably propagated. These findings have already been the subject of the patent application PCT/JP2013/073740, which is incorporated by reference herein.

As a result of intensive investigations, the present inventors also discovered that the efficiency in inducing a somatic embryo from callus varies with differences in the induction conditions during the induction of the callus from a tissue fragment from an isoprenoid-producing plant. That is, it was discovered that a somatic embryo is easily induced from callus that has been induced under certain culture conditions, while a somatic embryo is difficult to induce from callus that has been induced under certain culture conditions.

As a result of intensive investigations into the conditions for callus induction based on these findings, the following was discovered.

With respect to a method of inducing callus, including culturing a tissue fragment from an isoprenoid-producing plant in an induction medium containing a plant growth hormone and a carbon source to induce callus, it was discovered that when:

(1-1) the induction medium contains a metal ion-containing compound;

(1-2) the induction medium contains 2,4-dichlorophenoxyacetic acid and benzyladenine and the auxin plant hormone concentration in the induction medium is at least 1.2 mg/L;

(1-3) the induction medium contains a plant extract and the auxin plant hormone concentration in the induction medium is at least 1.2 mg/L; or

(1-4) the induction medium contains polyvinylpyrrolidone when the tissue fragment is a young leaf, a somatic embryo is then efficiently induced from the induced callus. That is, it was discovered that a somatic embryo is efficiently induced from callus that has been induced in accordance with any of (1-1) to (1-4), and as a result the callus can be stably regenerated into plants and, further, the plant can be stably propagated.

Thus, an aspect 1-1 of the present invention relates to a method of inducing callus, including culturing a tissue fragment from an isoprenoid-producing plant in an induction medium containing a plant growth hormone and a carbon source to induce callus, wherein the induction medium contains a metal ion-containing compound.

An aspect 1-2 of the present invention relates to a method of inducing callus, including culturing a tissue fragment from an isoprenoid-producing plant in an induction medium containing a plant growth hormone and a carbon source to induce callus, wherein the induction medium contains an auxin plant hormone and a cytokinin plant hormone as the plant growth hormone, the auxin plant hormone is 2,4-dichlorophenoxyacetic acid and the cytokinin plant hormone is benzyladenine, and the concentration of the auxin plant hormone in the induction medium is at least 1.2 mg/L. The induction medium preferably contains a metal ion-containing compound.

An aspect 1-3 of the present invention relates to a method of inducing callus, including culturing a tissue fragment from an isoprenoid-producing plant in an induction medium containing a plant growth hormone and a carbon source to induce callus, wherein the induction medium contains a plant extract, the induction medium contains an auxin plant hormone as the plant growth hormone, and the concentration of the auxin plant hormone in the induction medium is at least 1.2 mg/L.

An aspect 1-4 of the present invention relates to a method of inducing callus, including culturing a tissue fragment from an isoprenoid-producing plant in an induction medium containing a plant growth hormone and a carbon source to induce callus, wherein the tissue fragment is a young leaf and the induction medium contains polyvinylpyrrolidone.

In the aspects 1-1 to 1-4 of the present invention, the isoprenoid-producing plant is preferably a plant belonging to the family Euphorbiaceae, more preferably a plant belonging to the genus Hevea, and still more preferably Hevea brasiliensis.

In addition, as a result of intensive investigations, the present inventors discovered that the efficiency in inducing a somatic embryo from callus also varies with differences in the culture conditions for growing the induced callus. That is, it was discovered that a somatic embryo is easily induced from callus that has been grown under certain culture conditions, while a somatic embryo is difficult to induce from callus that has been grown under certain culture conditions.

As a result of intensive investigations into callus culture methods that grow callus, based on these findings, the following was discovered.

With respect to a method of culturing callus, including culturing callus from an isoprenoid-producing plant in a growth medium containing a plant growth hormone and a carbon source to grow the callus, it was discovered that when:

(2-1) the growth medium contains a metal ion-containing compound;

(2-2) the growth medium contains 2,4-dichlorophenoxyacetic acid and benzyladenine and the auxin plant hormone concentration in the growth medium is at least 1.2 mg/L; or

(2-3) the growth medium contains a gibberellin plant hormone,

a somatic embryo is then efficiently induced from the grown callus. That is, it was discovered that a somatic embryo is efficiently induced from callus grown in accordance with any of (2-1) to (2-3), and as a result the callus can be stably regenerated into plants and, further, the plant can be stably propagated.

Thus, an aspect 2-1 of the present invention relates to a method of culturing callus, including culturing callus from an isoprenoid-producing plant in a growth medium containing a plant growth hormone and a carbon source to grow the callus, wherein the growth medium contains a metal ion-containing compound.

An aspect 2-2 of the present invention relates to a method of culturing callus, including culturing callus from an isoprenoid-producing plant in a growth medium containing a plant growth hormone and a carbon source to grow the callus, wherein the growth medium contains an auxin plant hormone and a cytokinin plant hormone as the plant growth hormone, the auxin plant hormone is 2,4-dichlorophenoxyacetic acid and the cytokinin plant hormone is benzyladenine, and the concentration of the auxin plant hormone in the growth medium is at least 1.2 mg/L. The growth medium preferably contains a metal ion-containing compound.

An aspect 2-3 of the present invention relates to a method of culturing callus, including culturing callus from an isoprenoid-producing plant in a growth medium containing a plant growth hormone and a carbon source to grow the callus, wherein the growth medium contains a gibberellin plant hormone as the plant growth hormone.

In the aspects 2-1 to 2-3 of the present invention, the isoprenoid-producing plant is preferably a plant belonging to the family Euphorbiaceae, more preferably a plant belonging to the genus Hevea, and still more preferably Hevea brasiliensis.

As a result of intensive investigations, the present inventors additionally discovered that the ratio between the auxin plant hormone concentration and the gibberellin plant hormone concentration in the medium during the induction of a somatic embryo from callus is critical and that, by adjusting “the ratio of the auxin plant hormone concentration to the gibberellin plant hormone concentration” to at least a specific value, a somatic embryo is efficiently induced from callus, and as a result the callus can be stably regenerated into plants and, further, the plant can be stably propagated.

Thus, an aspect 3 of the present invention relates to a method of inducing a somatic embryo, including culturing callus from an isoprenoid-producing plant in a somatic embryo induction medium containing a plant growth hormone and a carbon source to form a somatic embryo, wherein the somatic embryo induction medium contains an auxin plant hormone and a gibberellin plant hormone as the plant growth hormone, and the ratio of the concentration of the auxin plant hormone to the concentration of the gibberellin plant hormone is at least 0.7. The callus from an isoprenoid-producing plant is preferably induced from an anther or a seed integument of an isoprenoid-producing plant.

In the aspect 3 of the present invention, the isoprenoid-producing plant is preferably a plant belonging to the family Euphorbiaceae, more preferably a plant belonging to the genus Hevea, and still more preferably Hevea brasiliensis.

A somatic embryo is efficiently induced from callus in accordance with the aspect 3 of the present invention and as a result the callus can be stably regenerated into plants.

Thus, an aspect 4-1 of the present invention relates to a method of regenerating a plant, including inducing a somatic embryo by the above-described somatic embryo induction method (aspect 3 of the present invention), followed by germination and rooting to regenerate the callus into plants.

When callus induced by the aspects 1-1 to 1-4 of the present invention or callus grown by the aspects 2-1 to 2-3 of the present invention is used here, the somatic embryo is efficiently induced and as a result the callus can be stably regenerated into plants.

Thus, an aspect 4-2 of the present invention relates to a method of regenerating a plant, including inducing a somatic embryo from callus induced by the above-described callus induction method (aspect 1-1 to 1-4 of the present invention) or callus grown by the above-described callus culture method (aspect 2-1 to 2-3 of the present invention), followed by germination and rooting to regenerate the callus into plants.

When callus induced in accordance with the aspect 1-1 to 1-4 of the present invention is used here, the somatic embryo is efficiently induced, and as a result the callus can be stably regenerated into plants and, further, the plant can be stably propagated.

Thus, an aspect 5 of the present invention relates to a method of propagating a plant, including inducing a somatic embryo from callus induced by the above-described callus induction method (aspect 1-1 to 1-4 of the present invention), followed by germination and rooting to propagate a plant.

Moreover, a somatic embryo is efficiently induced from callus in accordance with the aspect 3 of the present invention, and as a result the callus can be stably regenerated into plants and, further, the plant can be stably propagated.

Thus, a preferred embodiment of the aspect 5 of the present invention is a method of propagating a plant, including inducing a somatic embryo from callus induced by the above-described callus induction method (aspect 1-1 to 1-4 of the present invention) by the above-described somatic embryo induction method (aspect 3 of the present invention), followed by germination and rooting to propagate a plant.

Another preferred embodiment of the aspect 5 of the present invention is a method of propagating a plant, including growing callus induced by the above-described callus induction method (aspect 1-1 to 1-4 of the present invention) by the above-described callus culture method (aspect 2-1 to 2-3 of the present invention), and inducing a somatic embryo from the grown callus by the above-described somatic embryo induction method (aspect 3 of the present invention), followed by germination and rooting to propagate a plant.

As a result of intensive investigations, the present inventors also discovered that rooting can be induced by culturing a mature embryo of an isoprenoid-producing plant in a specific rooting medium.

Thus, an aspect 6 of the present invention relates to a method of inducing rooting in a mature embryo, including culturing a mature embryo of an isoprenoid-producing plant in a rooting medium containing a carbon source.

In the aspect 6 of the present invention, the rooting medium preferably contains a metal ion-containing compound and the rooting medium also preferably contains active carbon.

In the aspect 6 of the present invention, the isoprenoid-producing plant is preferably a plant belonging to the family Euphorbiaceae, more preferably a plant belonging to the genus Hevea, and still more preferably Hevea brasiliensis.

### Advantageous Effects of Invention

Using the callus induction method of the present invention (aspect 1-1 to 1-4 of the present invention), callus can be efficiently induced from a tissue fragment from an isoprenoid-producing plant and a somatic embryo can be efficiently induced from the resulting callus, and as a result the callus can be stably regenerated into plants and, further, the plant can be stably propagated.

Using the callus culture method of the present invention (aspect 2-1 to 2-3 of the present invention), callus can be efficiently grown and a somatic embryo can be efficiently induced from the grown callus, and as a result the callus can be stably regenerated into plants and, further, the plant can be stably propagated.

Using the somatic embryo induction method of the present invention (aspect 3 of the present invention), a somatic embryo can be efficiently induced from callus, and as a result the callus can be stably regenerated into plants and, further, the plant can be stably propagated.

Using the plant regeneration method of the present invention (aspect 4-1 and 4-2 of the present invention), callus can be stably regenerated into plants.

Using the plant propagation method of the present invention (aspect 5 of the present invention), a plant can be stably propagated, without being affected by e.g. weather and seasons, by carrying out tissue culture under controlled conditions.

Using the mature embryo rooting induction method of the present invention (aspect 6 of the present invention), rooting can be induced by culturing a mature embryo of an isoprenoid-producing plant in a specific rooting medium.

### BRIEF DESCRIPTION OF DRAWINGS

FIGS. 1A-1F are photographs that show, for Hevea brasiliensis, the appearance of callus formation, callus growth, somatic embryo formation, somatic embryo growth, and regeneration into plants; and

FIGS. 2A-2B are photographs that show examples of callus induced from tissue fragments from Hevea brasiliensis.

### DESCRIPTION OF EMBODIMENTS

Using the callus induction method of the present invention (aspect 1-1 to 1-4 of the present invention), callus can be efficiently induced from a tissue fragment from an isoprenoid-producing plant by culturing the tissue fragment from an isoprenoid-producing plant under the specific culture conditions (1-1) to (1-4), and a somatic embryo can be efficiently induced from the resulting callus, and as a result the callus can be stably regenerated into the plant and, further, the plant can be stably propagated.

Using the callus culture method of the present invention (aspect 2-1 to 2-3 of the present invention), callus can be efficiently grown by culturing callus from an isoprenoid-producing plant under the specific culture conditions (2-1) to (2-3), and a somatic embryo can be efficiently induced from the grown callus, and as a result the callus can be stably regenerated into plants and, further, the plant can be stably propagated.

Using the somatic embryo induction method of the present invention (aspect 3 of the present invention), a somatic embryo can be efficiently induced from callus by adjusting “the ratio of the auxin plant hormone concentration to the gibberellin plant hormone concentration” to at least a specific value, and as a result the callus can be stably regenerated into plants and, further, the plant can be stably propagated.

Using the plant regeneration method of the present invention (aspect 4-1 of the present invention), callus can be stably regenerated into plants by inducing a somatic embryo by the somatic embryo induction method (aspect 3 of the present invention), followed by germination and rooting. That is, as a result of the efficient induction of a somatic embryo from callus according to the aspect 3 of the present invention, the callus can be stably regenerated into plants.

Using the plant regeneration method of the present invention (aspect 4-2 of the present invention), a somatic embryo can be efficiently induced from callus induced in accordance with the aspect 1-1 to 1-4 of the present invention or callus grown in accordance with the aspect 2-1 to 2-3 of the present invention, and as a result the callus can be stably regenerated into plants.

Using the plant propagation method of the present invention (aspect 5 of the present invention), a plant can be stably propagated, without being affected by e.g. weather and seasons, by inducing a somatic embryo from callus induced by the callus induction method (aspect 1-1 to 1-4 of the present invention), followed by germination and rooting (the plant regeneration method of the aspect 4-2 of the present invention); more preferably by inducing a somatic embryo from callus induced by the callus induction method (aspect 1-1 to 1-4 of the present invention) by the somatic embryo induction method (aspect 3 of the present invention), followed by germination and rooting (the plant regeneration method of the aspect 4-1 of the present invention); and still more preferably by growing callus induced by the callus induction method (aspect 1-1 to 1-4 of the present invention) by the callus culture method (aspect 2-1 to 2-3 of the present invention), and inducing a somatic embryo from the grown callus by the somatic embryo induction method (aspect 3 of the present invention), followed by germination and rooting.

That is, as a result of the use of callus induced according to the aspect 1-1 to 1-4 of the present invention, or as a result of the efficient induction of a somatic embryo from callus according to the aspect 3 of the present invention in combination with the use of callus induced according to the aspect 1-1 to 1-4 of the present invention, the callus can be stably regenerated into plants and, further, the plant can be stably propagated. Specifically, a plant can be propagated stably in large quantities by inducing callus from a plant tissue fragment that can be acquired in large amounts, e.g., the stems or leaves of a plant, and regenerating the callus via somatic embryos into plants. Moreover, the plant can be propagated in larger quantities by proliferation of the somatic embryo.

Mutation is less likely to occur in regenerated plants than when they are maintained in the callus state (subculture) and plants can thus be stably provided. Moreover, since the regenerated plants can be grown in soil, unlike plant cells, e.g. callus, they do not require the expensive plant growth regulators required to maintain cells and thus costs can be kept down.

Using the mature embryo rooting induction method of the present invention (aspect 6 of the present invention), rooting can be induced by culturing a mature embryo of an isoprenoid-producing plant in a specific rooting medium. Specifically, rooting can be induced by culturing a somatic embryo from an isoprenoid-producing plant so that the somatic embryo becomes a matured somatic embryo (mature embryo), and culturing the mature embryo in a specific rooting medium; therefore, callus can be stably regenerated into plants. More specifically, rooting can be induced by inducing a somatic embryo from callus induced by the callus induction method (aspect 1-1 to 1-4 of the present invention), culturing the somatic embryo, and culturing the resulting mature embryo in a specific rooting medium; more preferably by inducing a somatic embryo from callus induced by the callus induction method (aspect 1-1 to 1-4 of the present invention) by the somatic embryo induction method (aspect 3 of the present invention), culturing the somatic embryo, and culturing the resulting mature embryo in a specific rooting medium; and still more preferably by growing callus induced by the callus induction method (aspect 1-1 to 1-4 of the present invention) by the callus culture method (aspect 2-1 to 2-3 of the present invention), inducing a somatic embryo from the grown callus by the somatic embryo induction method (aspect 3 of the present invention), culturing the somatic embryo, and culturing the resulting mature embryo in a specific rooting medium; therefore, callus can be stably regenerated into plants.

Thus, with the mature embryo rooting induction method (aspect 6 of the present invention), more suitable rooting can occur in the plant regeneration method (aspect 4-1 to 4-2 of the present invention) and in the plant propagation method (aspect 5 of the present invention).

In the present invention, callus denotes undifferentiated plant cells or a mass of undifferentiated plant cells. Also, in the present invention, somatic embryo denotes embryo-like tissue induced from callus. Also, in the present invention, a mature embryo denotes a matured somatic embryo, and the “mature” means that similar development to that of fertilized embryos occurs.

Isoprenoid-producing plants that can be used in the methods of the present invention are not particularly limited, provided that they are capable of producing isoprenoids. Examples include plants of the genus Hevea, e.g. Hevea brasiliensis; plants of the genus Sonchus, e.g. Sonchus oleraceus, Sonchus asper, Sonchus brachyotus, and Sonchus arvensis; plants of the genus Solidago, e.g. Solidago altissima, Solidago virgaurea subsp. asiatica, Solidago virgaurea subsp. leipcarpa, Solidago virgaurea subsp. leipcarpa f. paludosa, Solidago virga urea subsp. gigantea, and Solidago gigantea Ait. var. leiophylla Fernald; plants of the genus Helianthus, e.g. Helianthus annuus, Helianthus argophyllus, Helianthus atrorubens, Helianthus debilis, Helianthus decapetalus, and Helianthus giganteus; plants of the genus Taraxacum, e.g. dandelion (Taraxacum), Taraxacum venustum H. Koidz, Taraxacum hondoense Nakai, Taraxacum platycarpum Dahlst, Taraxacum japonicum, Taraxacum officinale Weber, and Taraxacum kok-saghyz; plants of the genus Ficus, e.g. Ficus carica, Ficus elastica, Ficus pumila L., Ficus erecta Thumb., Ficus ampelas Burm. f., Ficus benguetensis Merr., Ficus irisana Elm., Ficus microcarpa L. f., Ficus septica Burm. f., and Ficus benghalensis; guayule (Parhenium argentatum); and lettuce (Lactuca sativa). Preferred among the foregoing are plants belonging to the family Asteraceae, such as plants of the genera Sonchus, Solidago, Helianthus, and Taraxacum, and plants belonging to the family Euphorbiaceae, such as plants of the genus Hevea. Plants belonging to the genus Hevea are more preferred, and Hevea brasiliensis is still more preferred.

The plant propagation method of the present invention (aspect 5 of the present invention) is specifically described in the following. The plant propagation method of the present invention (aspect 5 of the present invention) basically uses the callus induction method of the present invention (aspect 1-1 to 1-4 of the present invention) and the plant regeneration method of the present invention (aspect 4-2 of the present invention). In a suitable embodiment, the plant propagation method further uses the callus culture method of the present invention (aspect 2-1 to 2-3 of the present invention) and the mature embryo rooting induction method of the present invention (aspect 6 of the present invention) in addition to the callus induction method of the present invention (aspect 1-1 to 1-4 of the present invention), the somatic embryo induction method of the present invention (aspect 3 of the present invention), and the plant regeneration method of the present invention (aspect 4-1 to 4-2 of the present invention). Thus, the description of the plant propagation method of the present invention (aspect 5 of the present invention) will also include the description of the callus induction methods of the present invention (aspects 1-1 to 1-4 of the present invention), the callus culture methods of the present invention (aspects 2-1 to 2-3 of the present invention), the somatic embryo induction method of the present invention (aspect 3 of the present invention), the plant regeneration methods of the present invention (aspects 4-1 to 4-2 of the present invention), and the mature embryo rooting induction method of the present invention (aspect 6 of the present invention).

The plant propagation method of the present invention (aspect 5 of the present invention) includes inducing a somatic embryo from callus induced by the callus induction method of the present invention (aspect 1-1 to 1-4 of the present invention), followed by germination and rooting (the plant regeneration method of the aspect 4-2 of the present invention) to propagate a plant.

A more suitable embodiment of the plant propagation method includes inducing a somatic embryo from callus induced by the callus induction method of the present invention (aspect 1-1 to 1-4 of the present invention) by the somatic embryo induction method of the present invention (aspect 3 of the present invention), followed by germination and rooting (the plant regeneration method of the aspect 4-1 or 4-2 of the present invention) to propagate a plant.

A still more suitable embodiment of the plant propagation method (aspect 5 of the present invention) includes growing callus induced by the callus induction method of the present invention (aspect 1-1 to 1-4 of the present invention) by the callus culture method of the present invention (aspect 2-1 to 2-3 of the present invention), and inducing a somatic embryo from the grown callus by the somatic embryo induction method of the present invention (aspect 3 of the present invention), followed by germination and rooting (the plant regeneration method of the aspect 4-1 or 4-2 of the present invention) to propagate a plant.

In a still more suitable embodiment, a mature embryo obtained by culturing the somatic embryo induced by the aforementioned method, is cultured in a specific rooting medium.

Each of the steps mentioned above is described in the following.

### (Induction Step)

In the induction step, callus is induced by culturing a tissue fragment from an isoprenoid-producing plant in an induction medium containing a plant growth hormone and a carbon source. The callus induction conditions are not particularly limited; however, by culturing a tissue fragment from an isoprenoid-producing plant under the specific culture conditions (1-1) to (1-4), callus can be efficiently induced from the tissue fragment from an isoprenoid-producing plant and a somatic embryo can be efficiently induced from the obtained callus, and as a result the callus can be stably regenerated into plants and, further, the plant can be stably propagated.

The tissue fragment is not particularly limited, but in specific terms it is preferably at least one selection from the group consisting of leaf, young leaf, petiole, stem, root, bud, petal, cotyledon, hypocotyl, anther, and seed. It is preferably an anther, a seed (preferably a seed integument), a leaf, a young leaf, a petiole, or a root, more preferably an anther or a seed (preferably a seed integument), among the foregoing, because they suitably provide callus that has high frequency of somatic embryo induction. The callus induced from an anther or a seed (preferably a seed integument) has particularly high frequency of somatic embryo induction.

In the specification, a young leaf denotes a young leaf that has sprouted from a new bud on a mature tree or sapling.

Also in the specification, a leaf denotes a leaf other than young leaf.

In the induction step, the surface of the tissue fragment from an isoprenoid-producing plant is first cleaned. When tissue taken from the plant interior is used as the tissue fragment, for example, cleaning may be carried out with an abrasive powder or with water containing approximately 0.1% of a surfactant. When, for example, a leaf is used, preferably the surface is cleaned with a soft sponge.

The tissue fragment is then disinfected or sterilized. This disinfection or sterilization can be carried out using known disinfectants or sterilizing agents, preferably ethanol, benzalkonium chloride, or an aqueous sodium hypochlorite solution.

Callus induction is then carried out by culturing the disinfected or sterilized tissue fragment in an induction medium containing a plant growth hormone and a carbon source. The induction medium may be a liquid or a solid, but solid culture is preferred because callus formation is facilitated by plating and culturing on the medium. When the induction medium is a liquid medium, static culture or shake culture may be carried out.

Examples of the plant growth hormone include auxin plant hormones, cytokinin plant hormones, and gibberellin plant hormones. Auxin plant hormones and cytokinin plant hormones are preferred thereamong.

The auxin plant hormones can be exemplified by 2,4-dichlorophenoxyacetic acid, naphthaleneacetic acid, indolebutyric acid, indoleacetic acid, indolepropionic acid, chlorophenoxyacetic acid, naphthoxyacetic acid, phenylacetic acid, 2,4,5-trichlorophenoxyacetic acid, para-chlorophenoxyacetic acid, 2-methyl-4-chlorophenoxyacetic acid, 4-fluorophenoxyacetic acid, 2-methoxy-3,6-dichlorobenzoic acid, 2-phenyl acid, picloram, and picolinic acid. Among the foregoing, 2,4-dichlorophenoxyacetic acid, naphthaleneacetic acid, indolebutyric acid, and indoleacetic acid are preferred; 2,4-dichlorophenoxyacetic acid or naphthaleneacetic acid is more preferred; and 2,4-dichlorophenoxyacetic acid is still more preferred.

The cytokinin plant hormones can be exemplified by benzyladenine, kinetin, zeatin, benzylaminopurine, isopentynylaminopurine, thidiazuron, isopentenyladenine, zeatin riboside, and dihydrozeatin. Among the foregoing, benzyladenine, kinetin, thidiazuron, and zeatin are preferred; benzyladenine or kinetin is more preferred; and benzyladenine is still more preferred.

The gibberellin plant hormones can be exemplified by gibberellins A₁ to A₁₃₆. Among these, gibberellin A₃ is preferred.

In the aspect 1-1 of the present invention, preferably 2,4-dichlorophenoxyacetic acid is used as the auxin plant hormone and benzyladenine is used as the cytokinin plant hormone.

In the aspect 1-2 of the present invention, 2,4-dichlorophenoxyacetic acid is used as the auxin plant hormone and benzyladenine is used as the cytokinin plant hormone.

In the aspect 1-3 of the present invention, preferably naphthaleneacetic acid and/or 2,4-dichlorophenoxyacetic acid is used as the auxin plant hormone and kinetin is used as the cytokinin plant hormone. More preferably, naphthaleneacetic acid and 2,4-dichlorophenoxyacetic acid are used as the auxin plant hormone and kinetin is used as the cytokinin plant hormone.

In the aspect 1-4 of the present invention, preferably benzyladenine and/or thidiazuron is used as the cytokinin plant hormone. More preferably, benzyladenine and thidiazuron are used as the cytokinin plant hormone. No auxin plant hormone is preferably used in the aspect 1-4 of the present invention.

There are no particular limitations on the carbon source, and examples include sugars such as sucrose, glucose, trehalose, fructose, lactose, galactose, xylose, allose, talose, gulose, altrose, mannose, idose, arabinose, apiose, maltose, mannitol, sorbitol, xylitol, erythritol, and so forth. Sucrose or glucose is preferred among the foregoing, with sucrose being more preferred.

The induction medium in the aspect 1-1 of the present invention contains a metal ion-containing compound. The metal ion, particularly silver ion, in the induction medium inhibits the action of ethylene on the tissue fragment and due to this callus that has high frequency of somatic embryo induction is suitably obtained.

Also in the aspects 1-2 to 1-4 of the present invention, particularly the aspect 1-2 of the present invention, the induction medium preferably contains a metal ion-containing compound. As a result, callus that has high frequency of somatic embryo induction is more suitably obtained.

The metal ion-containing compound may be any compound that contains a metal ion, such as salts of metal ions. The metal ion is not particularly limited, but ions of heavy metals, e.g. silver, iron, lead, gold, platinum, copper, chromium, cadmium, mercury, zinc, arsenic, manganese, cobalt, nickel, molybdenum, tungsten, tin, bismuth, uranium, and plutonium, are preferred and silver ions are more preferred. The form of the salt is not particularly limited, and examples include nitrates, sulfates, phosphates, acetates, carbonates, chlorates, iodates, perchlorates, chromates, bromates, chlorides, sulfides, azides, fluorides, oxides, iodides, cyanides, bromides, hydroxides, and other salts. Nitrates are preferred among the foregoing.

Silver ion-containing compounds can be exemplified by silver nitrate, silver sulfate, silver phosphate, silver acetate, silver carbonate, silver chlorate, silver iodate, silver perchlorate, silver chromate, silver bromate, silver chloride, silver sulfide, silver azide, silver fluoride, silver oxide, silver iodide, silver cyanide, silver bromide, and silver hydroxide. Silver nitrate is preferred among the foregoing because the effects of the present invention can be suitably achieved therewith.

The induction medium in the aspect 1-3 of the present invention contains a plant extract. As a result, callus that has high frequency of somatic embryo induction is suitably obtained.

Also in the aspects 1-1, 1-2, and 1-4 of the present invention, the induction medium preferably contains a plant extract. As a result, callus that has high frequency of somatic embryo induction is more suitably obtained.

The plant extract is not particularly limited, provided that it is extracted from a plant, preferably a fruit. Examples include coconut milk, banana extract, banana powder, potato extract, tomato juice, and so forth. Among the foregoing, liquid endosperm, preferably liquid endosperm derived from fruits, e.g. coconut milk (coconut water), can be suitably used.

The induction medium in the aspect 1-4 of the present invention contains polyvinylpyrrolidone. As a result, callus that has high frequency of somatic embryo induction is suitably obtained. The effect provided by the addition of polyvinylpyrrolidone to the induction medium is particularly large when the tissue fragment is a young leaf, but polyvinylpyrrolidone may be added to the induction medium also when other tissue fragments are used. Also in the aspects 1-1 to 1-3 of the present invention, the induction medium preferably contains polyvinylpyrrolidone. As a result, callus that has high frequency of somatic embryo induction is more suitably obtained.

The induction medium may be obtained by adding the plant growth hormone to any of base media such as basal media, e.g. White's medium (disclosed on pp. 20-36 of Shokubutsu Saibo Kogaku Nyumon (Introduction to Plant Cell Engineering), Japan Scientific Societies Press), Heller's medium (Heller R, Bot. Biol. Veg. Paris 14, 1-223 (1953)), SH medium (Schenk and Hildebrandt medium), MS medium (Murashige and Skoog medium) (disclosed on pp. 20-36 of Shokubutsu Saibo Kogaku Nyumon (Introduction to Plant Cell Engineering), Japan Scientific Societies Press), LS medium (Linsmaier and Skoog medium) (disclosed on pp. 20-36 of Shokubutsu Saibo Kogaku Nyumon (Introduction to Plant Cell Engineering), Japan Scientific Societies Press), Gamborg medium, B5 medium (disclosed on pp. 20-36 of Shokubutsu Saibo Kogaku Nyumon (Introduction to Plant Cell Engineering), Japan Scientific Societies Press), MB medium, and WP medium (Woody Plant: for woody plants) (the disclosures of the foregoing documents are incorporated by reference herein), and modified basal media obtained by altering the composition of the basal media. Among the foregoing, the induction medium is preferably MS medium, B5 medium, or WP medium, supplemented with the plant growth hormone. The induction medium also preferably contains an auxin plant hormone and a cytokinin plant hormone because this is suitable for callus maintenance and promotion of cell division.

To prepare the induction medium as a solid medium, the medium may be converted into a solid using a solidifying agent. There are no particular limitations on the solidifying agent, and examples include agar, gellan gum (e.g. Gelrite, Phytagel), agarose, gelatin, silica gel, and so forth.

The suitable composition and culture conditions of the induction medium vary depending on the type of plant and also vary depending on whether the medium is a liquid medium or a solid medium, but the composition is usually as follows (particularly in the case of plants belonging to the family Euphorbiaceae, preferably plants belonging to the genus Hevea, and particularly Hevea brasiliensis)).

In the aspects 1-1, 1-2, and 1-4 of the present invention, the carbon source concentration in the induction medium is preferably at least 0.1 mass %, more preferably at least 1 mass %, and still more preferably at least 2 mass %. The carbon source concentration is preferably not more than 10 mass %, more preferably not more than 6 mass %, and still more preferably not more than 4 mass %.

In the aspect 1-3 of the present invention, on the other hand, the carbon source concentration in the induction medium is preferably at least 0.1 mass %, more preferably at least 1 mass %, still more preferably at least 2 mass %, particularly preferably at least 3 mass %, and most preferably at least 5 mass %. The carbon source concentration is preferably not more than 10 mass %, and more preferably not more than 8 mass %.

In the specification, the carbon source concentration denotes the sugar concentration.

In the aspects 1-2 and 1-3 of the present invention, the auxin plant hormone concentration in the induction medium is at least 1.2 mg/L. As a result, callus that has high frequency of somatic embryo induction is suitably obtained. Also in the aspect 1-1 of the present invention, the auxin plant hormone concentration in the induction medium is preferably at least 1.2 mg/L. As a result, callus that has high frequency of somatic embryo induction is more suitably obtained.

In the aspects 1-1 to 1-3 of the present invention, the auxin plant hormone concentration in the induction medium is more preferably at least 1.4 mg/L, still more preferably at least 1.6 mg/L, and particularly preferably at least 1.8 mg/L. The auxin plant hormone concentration is preferably not more than 20 mg/L, more preferably not more than 10 mg/L, still more preferably not more than 4 mg/L, and particularly preferably not more than 2.5 mg/L.

In the aspect 1-4 of the present invention, on the other hand, the auxin plant hormone concentration is preferably not more than 0.1 mg/L, more preferably not more than 0.05 mg/L, and still more preferably not more than 0.01 mg/L.

The cytokinin plant hormone concentration in the induction medium is preferably at least 0 mg/L, more preferably at least 1×10⁻³ mg/L, still more preferably at least 0.1 mg/L, particularly preferably at least 0.5 mg/L, and most preferably at least 0.8 mg/L. The cytokinin plant hormone concentration is preferably not more than 15 mg/L, more preferably not more than 10 mg/L, still more preferably not more than 5 mg/L, and particularly preferably not more than 3 mg/L.

The metal ion-containing compound concentration in the induction medium is preferably at least 0.1 mg/L, more preferably at least 0.3 mg/L, still more preferably at least 0.5 mg/L, particularly preferably at least 0.7 mg/L, and most preferably at least 0.8 mg/L. The metal ion-containing compound concentration is preferably not more than 15 mg/L, more preferably not more than 10 mg/L, still more preferably not more than 5 mg/L, particularly preferably not more than 3 mg/L, and most preferably not more than 2 mg/L.

The plant extract concentration in the induction medium is preferably at least 0.1 mass %, more preferably at least 0.5 mass %, still more preferably at least 1 mass %, and particularly preferably at least 3 mass %. The plant extract concentration is preferably not more than 15 mass %, more preferably not more than 10 mass %, and still more preferably not more than 7 mass %.

The polyvinylpyrrolidone concentration in the induction medium is preferably at least 0.01 mass %, and more preferably at least 0.03 mass %. The polyvinylpyrrolidone concentration is preferably not more than 1 mass %, more preferably not more than 0.5 mass %, and still more preferably not more than 0.2 mass %.

The pH of the induction medium is preferably 4.0 to 10.0, more preferably 5.6 to 6.5, and still more preferably 5.7 to 5.8. The culture temperature is preferably 0° C. to 40° C., more preferably 20° C. to 35° C., and still more preferably 25° C. to 30° C. Culture may be carried out in the dark or in the light, but is preferably carried out in the dark. The culture time is not particularly limited, but culture for 1 to 10 weeks is preferred.

In the specification, the pH of the solid medium denotes the pH of the medium that incorporates all the components except the solidifying agent. Also in the specification, “in the dark” means an illuminance of 0 to 0.1 lx, while “in the light” means an illuminance greater than 0.1 lx.

When the induction medium is a solid medium, the solidifying agent concentration in the induction medium is preferably at least 0.1 mass %, and more preferably at least 0.2 mass %. The solidifying agent concentration is preferably not more than 3 mass %, more preferably not more than 1.1 mass %, and still more preferably not more than 0.8 mass %.

As described above, induction of callus can be carried out by culturing the disinfected or sterilized tissue fragment in the induction medium. The callus has high frequency of somatic embryo induction.

The induced callus may be genetically modified in the present invention. The introduction of recombinant genes may be carried out by commonly used methods under generally known conditions, and examples include, but are not limited to, the protoplast method, particle gun method, and Agrobacterium method (Seibutsu-Kagaku Jikken-ho 41, Shokubutsu Saibo Kogaku Nyumon (Biological and chemical experimental methods, vol. 41, Introduction to plant cell engineering), Sep. 1, 1998, Japan Scientific Societies Press, pp. 255-326; Plant Biotechnology, May 25, 2009, Saiwai Shobo, pp. 130-136, the disclosures of which are incorporated by reference herein).

The induced callus may be used directly in the somatic embryo induction step, which will be described later, but preferably the induced callus is first subjected to proliferation and the proliferated callus is used in the somatic embryo induction step because plant propagation in larger amounts can thereby be achieved. Callus proliferation may be carried out by culturing the callus under conditions capable of callus proliferation; for example, the callus can be proliferated (grown) by culturing the callus using similar medium composition and culture conditions to those in the induction step. The callus is preferably grown through the proliferation step described below, among other methods.

### (Proliferation Step)

In the proliferation step, for example, the callus induced in the induction step, optionally after genetic modification, for example as described above, is proliferated (grown) by culturing in a growth medium containing a plant growth hormone and a carbon source. There are no particular limitations on the conditions for growing the callus. However, by culturing the callus from an isoprenoid-producing plant under the specific culture conditions (2-1) to (2-3), the callus can be efficiently grown and a somatic embryo can be efficiently induced from the grown callus, and as a result the callus can be stably regenerated into plants and, further, the plant can be stably propagated.

The growth medium may be a liquid or a solid. When the growth medium is a liquid medium, static culture or shake culture may be carried out.

The plant growth hormone may be as described for the induction medium.

In the aspect 2-1 of the present invention, preferably 2,4-dichlorophenoxyacetic acid is used as the auxin plant hormone and benzyladenine is used as the cytokinin plant hormone.

In the aspect 2-2 of the present invention, 2,4-dichlorophenoxyacetic acid is used as the auxin plant hormone and benzyladenine is used as the cytokinin plant hormone.

In the aspect 2-3 of the present invention, a gibberellin plant hormone is used, and preferably an auxin plant hormone is used in addition to the gibberellin plant hormone. More preferably, naphthaleneacetic acid is used as the auxin plant hormone. Also in the aspect 2-3 of the present invention, a cytokinin plant hormone may be used in addition to the gibberellin plant hormone, and preferably benzyladenine or kinetin is used as the cytokinin plant hormone.

The growth medium in the aspect 2-1 of the present invention contains a metal ion-containing compound. The metal ion, particularly silver ion, in the growth medium inhibits the action of ethylene on the callus and due to this the callus that has high frequency of somatic embryo induction is suitably grown.

Also in the aspects 2-2 and 2-3 of the present invention, particularly the aspect 2-2 of the present invention, the growth medium preferably contains a metal ion-containing compound. As a result, the callus that has high frequency of somatic embryo induction is more suitably grown.

The growth medium may be obtained by adding the plant growth hormone to any of base media such as basal media, e.g. White's medium (disclosed on pp. 20-36 of Shokubutsu Saibo Kogaku Nyumon (Introduction to Plant Cell Engineering), Japan Scientific Societies Press), Heller's medium (Heller R, Bot. Biol. Veg. Paris 14, 1-223 (1953)), SH medium (Schenk and Hildebrandt medium), MS medium (Murashige and Skoog medium) (disclosed on pp. 20-36 of Shokubutsu Saibo Kogaku Nyumon (Introduction to Plant Cell Engineering), Japan Scientific Societies Press), LS medium (Linsmaier and Skoog medium) (disclosed on pp. 20-36 of Shokubutsu Saibo Kogaku Nyumon (Introduction to Plant Cell Engineering), Japan Scientific Societies Press), Gamborg medium, B5 medium (disclosed on pp. 20-36 of Shokubutsu Saibo Kogaku Nyumon (Introduction to Plant Cell Engineering), Japan Scientific Societies Press), MB medium, and WP medium (Woody Plant: for woody plants) (the disclosures of the foregoing documents are incorporated by reference herein), and modified basal media obtained by altering the composition of the basal media. Among the foregoing, the growth medium is preferably MS medium, B5 medium, or WP medium, supplemented with the plant growth hormone. The metal ion-containing compound, carbon source, and gibberellin plant hormone may suitably be as described for the induction medium.

To prepare the growth medium as a solid medium, the medium may be converted into a solid using a solidifying agent as described for the induction medium.

The suitable composition and culture conditions of the growth medium vary depending on the type of plant, and also vary depending on whether the medium is a liquid medium or a solid medium, but the composition is usually as follows (particularly in the case of plants belonging to the family Euphorbiaceae, preferably plants belonging to the genus Hevea, and particularly Hevea brasiliensis).

The carbon source concentration in the growth medium is preferably at least 0.1 mass %, more preferably at least 1 mass %, and still more preferably at least 2 mass %. The carbon source concentration is preferably not more than 10 mass %, more preferably not more than 6 mass %, and still more preferably not more than 4 mass %.

In the aspect 2-2 of the present invention, the auxin plant hormone concentration in the growth medium is at least 1.2 mg/L. As a result, the callus that has high frequency of somatic embryo induction can be suitably grown. Also in the aspect 2-1 of the present invention, the auxin plant hormone concentration in the growth medium is preferably at least 1.2 mg/L. As a result, the callus that has high frequency of somatic embryo induction can be more suitably grown.

In the aspects 2-1 and 2-2 of the present invention, the auxin plant hormone concentration in the growth medium is more preferably at least 1.4 mg/L, still more preferably at least 1.6 mg/L, and particularly preferably at least 1.8 mg/L. The auxin plant hormone concentration is preferably not more than 20 mg/L, more preferably not more than 10 mg/L, still more preferably not more than 4 mg/L, and particularly preferably not more than 2.5 mg/L.

In the aspect 2-3 of the present invention, on the other hand, the auxin plant hormone concentration is preferably 0.01 to 5 mg/L, more preferably 0.05 to 3 mg/L, and still more preferably 0.1 to 1 mg/L.

In the aspects 2-1 and 2-2 of the present invention, the cytokinin plant hormone concentration in the growth medium is preferably at least 0 mg/L, more preferably at least 1×10⁻³ mg/L, still more preferably at least 0.1 mg/L, particularly preferably at least 0.5 mg/L, most preferably at least 0.8 mg/L, and yet most preferably at least 1.5 mg/L. The cytokinin plant hormone concentration is preferably not more than 15 mg/L, more preferably not more than 10 mg/L, still more preferably not more than 5 mg/L, and particularly preferably not more than 3 mg/L.

In the aspect 2-3 of the present invention, the cytokinin plant hormone concentration in the growth medium is preferably 0 to 10 mg/L, wherein the upper limit is more preferably 7 mg/L, and still more preferably 5 mg/L.

In the aspect 2-3 of the present invention, the gibberellin plant hormone concentration in the growth medium is preferably 0.01 to 10 mg/L, and more preferably 0.03 to 5 mg/L, wherein the upper limit is still more preferably 3 mg/L, and particularly preferably 1 mg/L.

The metal ion-containing compound concentration in the growth medium is preferably at least 0.1 mg/L, more preferably at least 0.3 mg/L, still more preferably at least 0.5 mg/L, particularly preferably at least 0.7 mg/L, and most preferably at least 0.8 mg/L. The metal ion-containing compound concentration is preferably not more than 15 mg/L, more preferably not more than 10 mg/L, still more preferably not more than 5 mg/L, particularly preferably not more than 3 mg/L, and most preferably not more than 2 mg/L.

The pH of the growth medium is preferably 4.0 to 10.0, more preferably 5.6 to 6.5, and still more preferably 5.7 to 5.8. The culture temperature is preferably 0° C. to 40° C., more preferably 20° C. to 35° C., and still more preferably 25° C. to 30° C. Culture may be carried out in the dark or in the light, but is preferably carried out in the dark. The culture time is not particularly limited, but culture for 1 to 10 weeks is preferred.

When the growth medium is a solid medium, the solidifying agent concentration in the growth medium is preferably at least 0.1 mass %, and more preferably at least 0.2 mass %. The solidifying agent concentration is preferably not more than 3 mass %, and more preferably not more than 1.1 mass %.

As described above, the callus can be proliferated (grown) in the proliferation step by culturing the callus in the growth medium. The callus has high frequency of somatic embryo induction. The callus proliferated by the proliferation step is used in the subsequent somatic embryo induction step.

### (Somatic Embryo Induction Step)

In the somatic embryo induction step, a somatic embryo is formed by culturing the callus in a somatic embryo induction medium containing a plant growth hormone and a carbon source. A somatic embryo is induced (formed) from the callus, and the somatic embryo is cultured so that the somatic embryo becomes a matured somatic embryo (mature embryo); therefore, the callus can be stably regenerated into plants. Due to this, there are no particular limitations on the culture conditions in the somatic embryo induction step as long as the conditions can induce a somatic embryo from the callus. However, by adjusting “the ratio of the auxin plant hormone concentration to the gibberellin plant hormone concentration” to at least a specific value, a somatic embryo can be efficiently induced from the callus, and as a result the callus can be stably regenerated into plants and, further, the plant can be stably propagated.

In the somatic embryo induction step, for example, a somatic embryo is induced by culturing in the somatic embryo induction medium the callus induced in the induction step, optionally after genetic modification, for example as described above, and optionally after proliferation of the callus induced in the induction step in the proliferation step. The somatic embryo induction medium may be a liquid or a solid, but solid culture is preferred because somatic embryo induction is facilitated by plating and culturing on the medium. When the somatic embryo induction medium is a liquid medium, static culture or shake culture may be carried out.

The plant growth hormone may be as described for the induction medium, but an auxin plant hormone and a gibberellin plant hormone are used in the aspect 3 of the present invention. The auxin plant hormone is preferably indoleacetic acid and the gibberellin plant hormone is preferably gibberellin A₃.

The somatic embryo induction medium may be obtained by adding the plant growth hormone to any of base media such as the aforementioned basal media, and modified basal media obtained by altering the composition of the basal media. Among the foregoing, the somatic embryo induction medium is preferably MS medium, LS medium, B5 medium, or WP medium, supplemented with the plant growth hormone, more preferably MS medium supplemented with the plant growth hormone. The carbon source may suitably be as described for the induction medium.

To prepare the somatic embryo induction medium as a solid medium, the medium can be converted to a solid using a solidifying agent as described for the induction medium.

The suitable composition and culture conditions of the somatic embryo induction medium vary depending on the type of plant, and also vary depending on whether the medium is a liquid medium or a solid medium, but the composition is usually as follows (particularly in the case of plants belonging to the family Euphorbiaceae, preferably plants belonging to the genus Hevea, and particularly Hevea brasiliensis).

The ratio of the auxin plant hormone concentration to the gibberellin plant hormone concentration in the somatic embryo induction medium is at least 0.7, preferably at least 0.8, and more preferably at least 0.9. The ratio of the auxin plant hormone concentration to the gibberellin plant hormone concentration is preferably not more than 1.3, more preferably not more than 1.2, and still more preferably not more than 1.1. When the ratio of the auxin plant hormone concentration to the gibberellin plant hormone concentration is within the indicated range, a somatic embryo can be more efficiently induced from the callus, and as a result the callus can be more stably regenerated into plants and, further, the plant can be more stably propagated.

The auxin plant hormone concentration in the somatic embryo induction medium is preferably at least 0.1 mg/L, more preferably at least 0.5 mg/L, still more preferably at least 1 mg/L, particularly preferably at least 1.5 mg/L, and most preferably at least 1.8 mg/L. The auxin plant hormone concentration is preferably not more than 15 mg/L, more preferably not more than 8 mg/L, still more preferably not more than 5 mg/L, and particularly preferably not more than 3 mg/L.

The gibberellin plant hormone concentration in the somatic embryo induction medium is preferably at least 0.1 mg/L, more preferably at least 0.5 mg/L, still more preferably at least 1 mg/L, particularly preferably at least 1.5 mg/L, and most preferably at least 1.8 mg/L. The gibberellin plant hormone concentration is preferably not more than 15 mg/L, more preferably not more than 8 mg/L, still more preferably not more than 5 mg/L, and particularly preferably not more than 3 mg/L.

The carbon source concentration in the somatic embryo induction medium is preferably at least 0.1 mass %, more preferably at least 1 mass %, still more preferably at least 2 mass %, particularly preferably at least 3 mass %, and most preferably at least 6 mass %. The carbon source concentration is preferably not more than 10 mass %, and more preferably not more than 8 mass %.

The pH of the somatic embryo induction medium is not particularly limited, but is preferably 4.0 to 10.0, and more preferably 5.6 to 6.5. The culture temperature is preferably 0° C. to 40° C., more preferably 20° C. to 35° C., and still more preferably 25° C. to 32° C. Culture may be carried out in the dark or in the light, but culture is preferably carried out in the light for 12 to 20 hours per 24 hours, and the illuminance during culture in the light is preferably 2,000 to 25,000 lx. The culture time is not particularly limited, but culture for 4 to 48 weeks is preferred and culture for 6 to 24 weeks is more preferred.

When the somatic embryo induction medium is a solid medium, the solidifying agent concentration in the somatic embryo induction medium is preferably at least 0.1 mass %, and more preferably at least 0.15 mass %. The solidifying agent concentration is preferably not more than 3 mass %, and more preferably not more than 1.1 mass %.

As described above, a somatic embryo can be formed in the somatic embryo induction step by culturing the callus in the somatic embryo induction medium. The somatic embryo formed in the somatic embryo induction step is further cultured so that it matures into a mature embryo.

### (Somatic Embryo Maturation Step)

In the somatic embryo maturation step, the somatic embryo formed as above is cultured in a maturation medium to mature the somatic embryo.

In the somatic embryo maturation step, for example, the somatic embryo is matured by culturing the somatic embryo formed in the somatic embryo induction step in the maturation medium. The maturation medium may be a liquid or a solid, but solid culture is preferred because maturation of the somatic embryo is facilitated by plating and culturing on the medium. When the maturation medium is a liquid medium, static culture or shake culture may be carried out.

The maturation medium may be, for example, any of the aforementioned basal media, and modified basal media obtained by altering the composition of the basal media. However, the maturation medium may suitably have a similar composition to that of the somatic embryo induction medium because the somatic embryo can thereby be suitably matured.

When the maturation medium is used as a solid medium, the medium may be converted into a solid using a solidifying agent as described for the induction medium.

The suitable culture conditions in the somatic embryo maturation step vary depending on the type of plant, and also vary depending on whether the medium is a liquid medium or a solid medium, but the conditions are usually as follows (particularly in the case of plants belonging to the family Euphorbiaceae, preferably plants belonging to the genus Hevea, and particularly Hevea brasiliensis).

The pH of the maturation medium is not particularly limited, but is preferably 4.0 to 10.0, and more preferably 5.6 to 6.5. The culture temperature is preferably 0° C. to 40° C., more preferably 20° C. to 36° C., and still more preferably 20° C. to 30° C. Culture may be carried out in the dark or in the light, but culture is preferably carried out in the light for 10 to 16 hours per 24 hours, and the illuminance during culture in the light is preferably 2,000 to 25,000 lx. The culture time is not particularly limited, but culture for 4 to 16 weeks is preferred.

When the maturation medium is a solid medium, the solidifying agent concentration in the maturation medium is preferably at least 0.1 mass %, and more preferably at least 0.2 mass %. The solidifying agent concentration is preferably not more than 2 mass %, and more preferably not more than 1.1 mass %.

As described above, the somatic embryo can be matured in the somatic embryo maturation step by culturing the formed somatic embryo in the maturation medium. In the somatic embryo maturation step, not only does the somatic embryo mature, but germination may also occur. By culturing the somatic embryo in the maturation medium in the somatic embryo maturation step, the somatic embryo matures and then germination is observed. The somatic embryo matured in the somatic embryo maturation step is used in the subsequent rooting step. The timing of transfer to the subsequent rooting step is preferably after germination occurs. The somatic embryo maturation step may be omitted when the somatic embryo has already matured in the somatic embryo induction step.

### (Rooting Step)

In the rooting step, rooting is induced by culturing the mature embryo in a rooting medium containing a carbon source. The mature embryo used may be a mature embryo obtained in the somatic embryo maturation step, or the mature embryo that has matured in the somatic embryo induction step may be directly used in this step.

In the rooting step, for example, rooting is induced by culturing the mature embryo obtained in the somatic embryo maturation step in the rooting medium. The rooting medium may be a liquid or a solid, but solid culture is preferred because rooting induction is facilitated by plating and culturing on the medium. When the rooting medium is a liquid medium, static culture or shake culture may be carried out.

The rooting medium may be, for example, any of the aforementioned basal media, and modified basal media obtained by altering the composition of the basal media. However, in the case of plants belonging to the family Euphorbiaceae, preferably plants belonging to the genus Hevea, and particularly Hevea brasiliensis, the rooting medium is preferably MS medium or modified MS medium obtained by altering the composition of MS medium, more preferably ½MS medium, because they allow for suitable rooting. The rooting medium, particularly ½MS medium, may or may not contain a plant growth hormone, but preferably contains a plant growth hormone. The plant growth hormone may be as described for the induction medium. Preferably, the rooting medium contains an auxin plant hormone, more preferably 2,4-dichlorophenoxyacetic acid, naphthaleneacetic acid, indolebutyric acid, or indoleacetic acid, and particularly preferably indolebutyric acid.

The rooting medium preferably contains a metal ion-containing compound. As a result, rooting can be more suitably induced. The metal ion-containing compound may suitably be as described for the induction medium.

The rooting medium preferably contains active carbon. This prevents accumulation of substances that inhibit tissue growth, and as a result rooting can be more suitably induced.

The carbon source may suitably be as described for the induction medium. The composition of the rooting medium may be the same as the composition of the maturation medium. The rooting step may be omitted when rooting has already occurred in the somatic embryo induction step or the somatic embryo maturation step.

When the rooting medium is used as a solid medium, the medium can be converted into a solid using a solidifying agent as described for the induction medium.

The suitable composition and culture conditions of the rooting medium vary depending on the type of plant, and also vary depending on whether the medium is a liquid medium or a solid medium, but the conditions are usually as follows (particularly in the case of plants belonging to the family Euphorbiaceae, preferably plants belonging to the genus Hevea, and particularly Hevea brasiliensis).

The auxin plant hormone concentration in the rooting medium is preferably 0.5 to 15 mg/L, more preferably 1 to 10 mg/L, and still more preferably 3 to 6 mg/L.

The cytokinin plant hormone concentration in the rooting medium is preferably 0 to 7 mg/L. The upper limit is preferably 5 mg/L, more preferably 1 mg/L, and still more preferably 0.01 mg/L, and particularly preferably substantially no cytokinin plant hormone is present (0 mg/L).

The carbon source concentration in the rooting medium is preferably at least 0.1 mass %, more preferably at least 1 mass %, and still more preferably at least 2 mass %. The carbon source concentration is preferably not more than 10 mass %, more preferably not more than 8 mass %, and still more preferably not more than 5 mass %.

The active carbon concentration in the rooting medium is preferably at least 0.01 mass %, more preferably at least 0.02 mass %, and still more preferably at least 0.03 mass %. The active carbon concentration is preferably not more than 1 mass %, more preferably not more than 0.1 mass %, and still more preferably not more than 0.07 mass %.

The metal ion-containing compound concentration in the rooting medium is preferably at least 0.1 mg/L, more preferably at least 0.3 mg/L, still more preferably at least 0.5 mg/L, particularly preferably at least 0.7 mg/L, and most preferably at least 0.8 mg/L. The metal ion-containing compound concentration is preferably not more than 15 mg/L, more preferably not more than 10 mg/L, still more preferably not more than 5 mg/L, particularly preferably not more than 3 mg/L, and most preferably not more than 2 mg/L.

The pH of the rooting medium is not particularly limited, but is preferably 4.0 to 10.0, more preferably 5.5 to 6.5, and still more preferably 5.6 to 6.0. The culture temperature is preferably 0° C. to 40° C., more preferably 20° C. to 35° C., and still more preferably 25° C. to 32° C.

Culture may be carried out in the dark or in the light, but culture is preferably carried out in the light for 14 to 16 hours per 24 hours, and the illuminance during culture in the light is preferably 2,000 to 25,000 lx. The culture time is not particularly limited, but culture for 4 to 10 weeks is preferred.

When the rooting medium is a solid medium, the solidifying agent concentration in the rooting medium is preferably at least 0.1 mass %, more preferably at least 0.2 mass %, and still more preferably at least 0.3 mass %. The solidifying agent concentration is preferably not more than 2 mass %, more preferably not more than 1.1 mass %, and still more preferably not more than 0.8 mass %.

As described above, rooting can be induced in the rooting step by culturing the mature embryo in the rooting medium, thereby obtaining the rooted mature embryo (young plant). This young plant may be directly transplanted to soil, but is preferably transplanted to soil after transfer to an artificial soil, e.g. vermiculite, and then acclimatization.

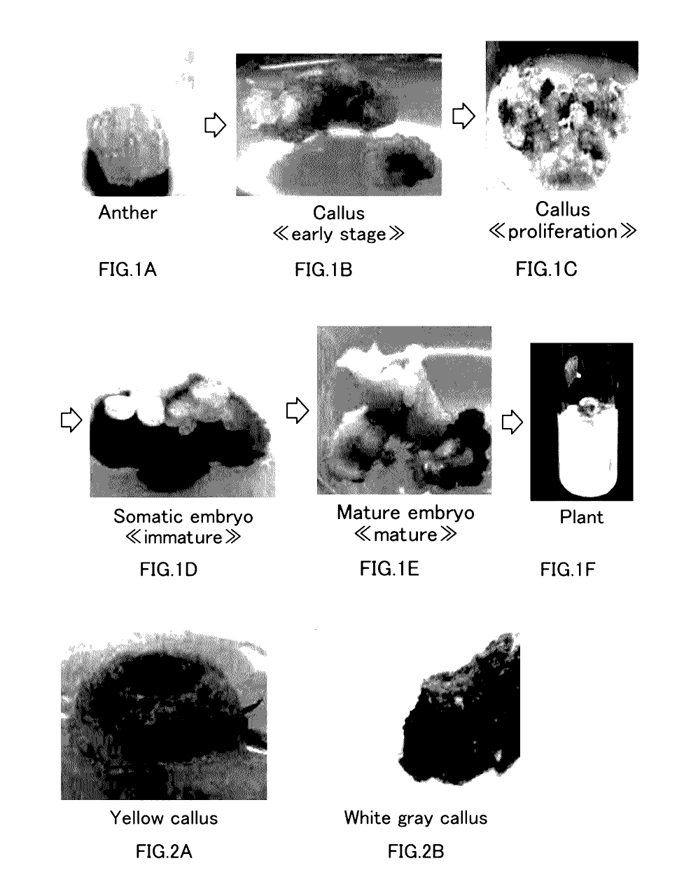

As has been described above, callus can be stably regenerated into plants by inducing a somatic embryo from the callus and culturing the somatic embryo according to the present invention. Further, a plant can be stably propagated, without being affected by e.g. weather and seasons, by performing tissue culture in a controlled environment. FIG. 1 shows examples of the appearance of induction of callus from an anther, growth of the induced callus, somatic embryo induction and maturation into a mature embryo, and regeneration into plants.

### Examples

The present invention is specifically described with reference to examples, but the present invention is not limited only to these.

The individual reagents used in the examples with Hevea brasiliensis are collectively described in the following.

NAA: naphthaleneacetic acid
2,4-D: 2,4-dichlorophenoxyacetic acid
BA: benzyladenine
KI: kinetin
IAA: indoleacetic acid
IBA: indolebutyric acid
GA3: gibberellin A₃
TDZ: thidiazuron
PVP: polyvinylpyrrolidone
Gelling agent: agar
Hevea brasiliensis: Hevea brasiliensis indigenous to Prince of Songkla University

### (Callus Induction (Induction Step))

The tissue fragments indicated in Table 1 were collected from the Hevea brasiliensis. Next, the surface of each collected tissue fragment was washed with running water and then 70% ethanol, subsequently sterilized with a sodium hypochlorite solution diluted at approximately 5 to 10%, and washed again with running water.

Each of the sterilized tissue fragments was then inserted into an induction medium (solid medium) and cultured (induction step). The composition of the induction media used for callus formation from the Hevea brasiliensis is shown in Table 1. The induction media were prepared by adding the components indicated in Table 1 to MS medium (disclosed on pp. 20-36 of Shokubutsu Saibo Kogaku Nyumon (Introduction to Plant Cell Engineering), Japan Scientific Societies Press), adjusting the pH of the medium to 5.7 to 5.8, adding thereto 0.75 mass % of the gelling agent, sterilizing in an autoclave (121° C., 20 minutes), and cooling in a clean bench.

The Hevea brasiliensis tissue fragments were inserted into the induction media (solid media) and cultured for 4 weeks in the dark at a culture temperature of 28° C. to induce callus (undifferentiated cells) from the Hevea brasiliensis tissue fragments.

After the 4-week culture in the induction media, callus induction was checked visually and evaluated using the criteria given below. The results are shown in Table 1.

Good: Callus formation was observed for at least 50% of the tissue fragment
Poor: No callus formation or death

TABLE 1

| | | | | | | | | Coconut | | | | | | | |
|---|---|---|---|---|---|---|---|---|---|---|---|---|---|---|---|
| | | | | | | | Silver | water | Sucrose | PVP | Gelling | | | | |
| | | NAA | 2,4-D | BA | KI | TDZ | nitrate | Conc. | Conc. | Conc. | agent | Tem- | Day | Callus | |
| | Source | Conc. | Conc. | Conc. | Conc. | Conc. | Conc. | (mass | (mass | (mass | Conc. | perature | length | for- | Callus |
| No | tissue | (mg/L) | (mg/L) | (mg/L) | (mg/L) | (mg/L) | (mg/L) | %) | %) | %) | (%) | (° C. ) | (hr) | mation | appearance |
| Ex. 1 | Anther | 1 | 1 | — | 1 | — | — | 5 | 7 | — | 0.75 | 28 | 0 | Good | Brit- |
| | | | | | | | | | | | | | (dark) | | tle/Yellow |
| Ex. 2 | Integ- | — | 2 | 2 | — | — | — | — | 3 | — | 0.75 | 28 | 0 | Good | Brit- |
| | ument | | | | | | | | | | | | (dark) | | tle/Yellow |
| Ex. 3 | Young | — | — | 0.5 | — | 0.5 | — | — | 3 | 0.05 | 0.75 | 28 | 0 | Good | Com- |
| | leaf | | | | | | | | | | | | (dark) | | pact/White |
| | | | | | | | | | | | | | | | gray |
| Ex. 4 | Petiole | — | 2 | 2 | — | — | 1 | — | 3 | — | 0.75 | 28 | 0 | Good | Com- |
| | | | | | | | | | | | | | (dark) | | pact/White |
| | | | | | | | | | | | | | | | gray |
| Ex. 5 | Leaf | — | 2 | 2 | — | — | 1 | — | 3 | — | 0.75 | 28 | 0 | Good | Com- |
| | | | | | | | | | | | | | (dark) | | pact/White |
| | | | | | | | | | | | | | | | gray |
| Ex. 6 | Root | — | 2 | 2 | — | — | 1 | — | 3 | — | 0.75 | 28 | 0 | Good | Com- |
| | | | | | | | | | | | | | (dark) | | pact/White |
| | | | | | | | | | | | | | | | gray |
| Ex. 7 | Integ- | — | 2 | 2 | — | — | 1 | — | 3 | — | 0.75 | 28 | 0 | Good | Brit- |
| | ument | | | | | | | | | | | | (dark) | | tle/Yellow |
| Com. | Anther | 0.5 | — | 0.5 | — | — | — | 5 | 2 | — | 0.75 | 28 | 0 | Poor | — |
| Ex. 1 | | | | | | | | | | | | | (dark) | | |
| Com. | Integ- | 0.5 | 0.5 | 1 | — | — | — | — | 3 | — | 0.75 | 28 | 0 | Poor | — |
| Ex. 2 | ument | | | | | | | | | | | | (dark) | | |
| Com. | Leaf | 0.5 | 0.5 | 1 | — | — | — | — | 3 | — | 0.75 | 28 | 0 | Poor | — |
| Ex. 3 | | | | | | | | | | | | | (dark) | | |
| Com. | Root | 0.5 | — | 0.5 | — | — | — | 5 | 2 | — | 0.75 | 28 | 0 | Poor | — |
| Ex. 4 | | | | | | | | | | | | | (dark) | | |

The results in Table 1 demonstrated that callus was efficiently induced from tissue fragments from an isoprenoid-producing plant (Hevea brasiliensis) by culturing the tissue fragments from an isoprenoid-producing plant (Hevea brasiliensis) under the specific culture conditions (1-1) to (1-4) described above. It was also demonstrated that the obtained callus had high frequency of somatic embryo induction, i.e., a somatic embryo was efficiently induced from the obtained callus, and as a result the callus was stably regenerated into plants. In particular, the calli induced according to Examples 4 to 7, in which the induction medium contained a metal ion-containing compound, had high frequency of somatic embryo induction. FIG. 2 is photographs that show examples of callus induced from tissue fragments from Hevea brasiliensis.

In Table 1, Examples 4 to 7 correspond to embodiments of the aspect 1-1 of the present invention; Examples 2 and 4 to 7 correspond to embodiments of the aspect 1-2 of the present invention; Example 1 corresponds to an embodiment of the aspect 1-3 of the present invention; and Example 3 corresponds to an embodiment of the aspect 1-4 of the present invention.

Next, each induced callus (the calli induced under the conditions in Examples 1, 7, 4, 5, and 6 were used in examples in Table 2 in which the source tissue was the anther, integument, petiole, leaf, and root, respectively; a callus induced under the same conditions as in Example 4 using the stem as the tissue fragment was used in an example in Table 2 in which the source tissue was the stem) was subcultured in a growth medium (solid medium) approximately every month to proliferate the callus (proliferation step). The composition of the growth media is shown in Table 2.

The growth media were prepared by adding the components indicated in Table 2 to MS medium (disclosed on pp. 20-36 of Shokubutsu Saibo Kogaku Nyumon (Introduction to Plant Cell Engineering), Japan Scientific Societies Press), adjusting the pH of the medium to 5.7 to 5.8, adding thereto 0.75 mass % of the gelling agent, sterilizing in an autoclave (121° C., 20 minutes), and cooling in a clean bench.

Culture was carried out in the dark (0 to 0.1 lx) or light (10,000 lx, day length indicated in Table 2) to stimulate callus growth.

After 4-week culture in the growth media, callus growth was checked visually and evaluated using the following criteria. The results are shown in Table 2.

Good: Proliferation was observed for more than 50% of the callus
Acceptable: Proliferation was observed for not more than 50% of the callus
Poor: No callus proliferation or death

TABLE 2

| | | NAA | 2,4-D | GA3 | BA | KI | Silver nitrate | Sucrose | Gelling agent | | | |
|---|---|---|---|---|---|---|---|---|---|---|---|---|
| | | Concen- | Concen- | Concen- | Concen- | Concen- | Concen- | Concen- | Concen- | Tem- | Day | Callus |
| | Source | tration | tration | tration | tration | tration | tration | tration | tration | perature | length | prolif- |
| No | tissue | (mg/L) | (mg/L) | (mg/L) | (mg/L) | (mg/L) | (mg/L) | (mass %) | (%) | (° C. ) | (hr) | eration |
| Ex. 8 | Anther | 0.3 | — | 0.5 | — | — | — | 7 | 0.75 | 28 | 0 (dark) | Good |
| Ex. 9 | Integument | 0.2 | — | 0.05 | 1 | 3 | — | 3 | 0.75 | 28 | 0 (dark) | Good |
| Ex. 10 | Petiole | — | 2 | — | 2 | — | — | 3 | 0.75 | 28 | 0 (dark) | Acceptable |
| Ex. 11 | Stem | — | 2 | — | 2 | — | 1 | 3 | 0.75 | 28 | 0 (dark) | Good |
| Ex. 12 | Leaf | — | 2 | — | 2 | — | 1 | 3 | 0.75 | 28 | 0 (dark) | Good |
| Ex. 14 | Petiole | — | 2 | — | 2 | — | 1 | 3 | 0.75 | 28 | 0 (dark) | Good |
| Ex. 15 | Integument | — | 2 | — | 2 | | 1 | 3 | 0.75 | 28 | 0 (dark) | Good |
| Com. | Anther | — | 0.8 | — | 2 | 1 | — | 7 | 0.75 | 28 | 12 | Poor |
| Ex. 5 | | | | | | | | | | | | |
| Com. | Integument | — | 0.8 | — | 2 | 1 | — | 7 | 0.75 | 28 | 0 (dark) | Poor |
| Ex. 6 | | | | | | | | | | | | |
| Com. | Leaf | — | 0.8 | — | 2 | 1 | — | 7 | 0.75 | 28 | 0 (dark) | Poor |
| Ex. 7 | | | | | | | | | | | | |
| Com. | Root | — | 0.8 | — | 2 | 1 | — | 7 | 0.75 | 28 | 12 | Poor |
| Ex. 8 | | | | | | | | | | | | |

The results in Table 2 demonstrated that callus from an isoprenoid-producing plant (Hevea brasiliensis) was efficiently grown by culturing the callus under the specific culture conditions (2-1) to (2-3). It was also demonstrated that the grown callus had high frequency of somatic embryo induction, i.e., a somatic embryo was efficiently induced from the grown callus, and as a result the callus was stably regenerated into plants. In particular, the calli grown according to Examples 11 to 15, in which the growth medium contained a metal ion-containing compound, had high frequency of somatic embryo induction.

In Table 2, Examples 11 to 15 correspond to embodiments of the aspect 2-1 of the present invention; Examples 10 to 15 correspond to embodiments of the aspect 2-2 of the present invention; and Examples 8 and 9 correspond to embodiments of the aspect 2-3 of the present invention.

### (Study of Medium for Somatic Embryo Formation (Somatic Embryo Induction Step))

Next, using the induced callus (the calli induced under the conditions in Examples 1 and 7 were used in examples in Table 3 in which the source tissue was the anther and integument, respectively) and MS medium (basal medium), a study was performed into the medium (somatic embryo induction medium) conditions for inducing somatic embryo formation from callus (see Table 3). The pH was adjusted to 5.7 to 5.8. The gelling agent (Phytagel), which corresponds to a solidifying agent, was added at 0.75 mass % to the medium. Using the thus prepared solid media (after sterilization), the callus induced in the induction step was cultured for 1 to 3 months under a light cycle of 16 hours light (10,000 lx) per 24 hours at a culture temperature of 28° C. Each medium was exchanged every 1 month of the culture.

After the 1 to 3-month culture in the somatic embryo induction medium, somatic embryo formation was checked visually and evaluated using the following criteria. The results are shown in Table 3.

Good: Somatic embryo formation was observed for 10% of the callus
Poor: No somatic embryo formation or death

TABLE 3

| | | IAA | NAA | GA3 | BA | Sucrose | Gelling agent | | | Somatic |
|---|---|---|---|---|---|---|---|---|---|---|
| | | Concen- | Concen- | Concen- | Concen- | Concen- | Concen- | Tem- | Day | embryo |
| | Source | tration | tration | tration | tration | tration | tration | perature | length | for- |
| No | tissue | (mg/L) | (mg/L) | (mg/L) | (mg/L) | (mass %) | (%) | (° C. ) | (hr) | mation |
| Ex. 16 | Anther | 2 | — | 2 | — | 7 | 0.75 | 28 | 16 | Good |
| Ex. 17 | Integument | 2 | — | 2 | — | 7 | 0.75 | 28 | 16 | Good |
| Com. | Anther | — | 2 | — | 2 | 3 | 0.75 | 28 | 12 | Poor |
| Ex. 9 | | | | | | | | | | |
| Com. | Integument | — | 2 | — | 2 | 3 | 0.75 | 28 | 12 | Poor |
| Ex. 10 | | | | | | | | | | |

### (Somatic Embryo Maturation (Somatic Embryo Maturation Step))

Next, the formed somatic embryo was subcultured in a medium having the same composition as the somatic embryo induction medium in order to mature the somatic embryo. Culture was carried out for 8 weeks at a culture temperature of 25° C. under a light cycle of 12 hours light (10,000 lx) per 24 hours. The somatic embryo matured well into a mature embryo, and germination was also observed.

Thus, for the media where somatic embryo formation had occurred, after somatic embryo formation the somatic embryo matured into a mature embryo and germination was also observed. In contrast, for the media where somatic embryo formation had not occurred, no mature embryo was formed even when culture was continued as such. This demonstrated that a mature embryo is stably formed through the induction of a somatic embryo from callus.

### (Rooting (Rooting Step))

Next, the mature embryo grown to about 3 cm was transplanted to a rooting medium in order to induce rooting.

The composition of the rooting medium is given in Table 4. The rooting medium was prepared by adding the components indicated in Table 4 to ½MS medium (disclosed on pp. 20-36 of Shokubutsu Saibo Kogaku Nyumon (Introduction to Plant Cell Engineering), Japan Scientific Societies Press), adjusting the pH of the medium to 5.7 to 5.8, adding thereto 0.75 mass % of the gelling agent, sterilizing in an autoclave (121° C., 20 minutes), and cooling in a clean bench.

The mature embryo grown to about 3 cm was inserted into the rooting medium (solid medium) and cultured for 8 weeks under a light cycle of 16 hours light (10,000 lx) per 24 hours at a culture temperature of 28° C. In Example 18 using the specific rooting medium, after culture in this medium good rooting was observed and a young plant was obtained. Here, transplantation to the same medium was performed every 4 weeks. In contrast, in Comparative Example 11 using no specific rooting medium, rooting was not observed, and the somatic embryo was exhausted when culture was continued.

TABLE 4

| | | IBA | AgNO₃ | Sucrose | Active carbon | Gelling agent | |
|---|---|---|---|---|---|---|---|
| | Temperature | Concentration | Concentration | Concentration | Concentration | Concentration | |
| No | (° C. ) | (mg/L) | (mg/L) | (mass %) | (mass %) | (%) | Rooting |
| Ex. 18 | 28 | 5 | 1 | 3 | 0.05 | 0.75 | Observed |
| Com. | 28 | — | — | 3 | — | 0.75 | Not observed |
| Ex. 11 | | | | | | | |

1. A method of inducing callus, comprising culturing a tissue fragment from an isoprenoid-producing plant in an induction medium containing a plant growth hormone and a carbon source to induce callus, wherein
the induction medium contains a metal ion-containing compound.

2. A method of inducing callus, comprising culturing a tissue fragment from an isoprenoid-producing plant in an induction medium containing a plant growth hormone and a carbon source to induce callus, wherein
the induction medium contains an auxin plant hormone and a cytokinin plant hormone as the plant growth hormone,
the auxin plant hormone is 2,4-dichlorophenoxyacetic acid and the cytokinin plant hormone is benzyladenine, and
the concentration of the auxin plant hormone in the induction medium is at least 1.2 mg/L.

3. A method of inducing callus, comprising culturing a tissue fragment from an isoprenoid-producing plant in an induction medium containing a plant growth hormone and a carbon source to induce callus, wherein
the induction medium contains a plant extract,
the induction medium contains an auxin plant hormone as the plant growth hormone, and
the concentration of the auxin plant hormone in the induction medium is at least 1.2 mg/L.

4. A method of inducing callus, comprising culturing a tissue fragment from an isoprenoid-producing plant in an induction medium containing a plant growth hormone and a carbon source to induce callus, wherein
the tissue fragment is a young leaf, and
the induction medium contains polyvinylpyrrolidone.

5. A method of culturing callus, comprising culturing callus from an isoprenoid-producing plant in a growth medium containing a plant growth hormone and a carbon source to grow the callus, wherein
the growth medium contains a metal ion-containing compound.

6. A method of culturing callus, comprising culturing callus from an isoprenoid-producing plant in a growth medium containing a plant growth hormone and a carbon source to grow the callus, wherein
the growth medium contains an auxin plant hormone and a cytokinin plant hormone as the plant growth hormone,
the auxin plant hormone is 2,4-dichlorophenoxyacetic acid and the cytokinin plant hormone is benzyladenine, and
the concentration of the auxin plant hormone in the growth medium is at least 1.2 mg/L.

7. A method of culturing callus, comprising culturing callus from an isoprenoid-producing plant in a growth medium containing a plant growth hormone and a carbon source to grow the callus, wherein
the growth medium contains a gibberellin plant hormone as the plant growth hormone.

8. A method of inducing a somatic embryo, comprising culturing callus from an isoprenoid-producing plant in a somatic embryo induction medium containing a plant growth hormone and a carbon source to form a somatic embryo, wherein
the somatic embryo induction medium contains an auxin plant hormone and a gibberellin plant hormone as the plant growth hormone, and
the ratio of the concentration of the auxin plant hormone to the concentration of the gibberellin plant hormone is at least 0.7.

9. A method of inducing rooting in a mature embryo, comprising culturing a mature embryo of an isoprenoid-producing plant in a rooting medium containing a carbon source to induce rooting.
